(19) United States
(12) Patent Application Publication
(10) Pub. No.: US 2019/0014758 A1
(43) Pub. Date: Jan. 17, 2019

(54) HUMANIZED MOUSE MODEL

(71) Applicant: CytoDyn Inc., Vancouver, WA (US)

(72) Inventor: Denis R. Burger, Vancouver, WA (US)

(21) Appl. No.: 15/977,933

(22) Filed: May 11, 2018

Related U.S. Application Data
(60) Provisional application No. 62/585,974, filed on Nov. 14, 2017.
(60) Provisional application No. 62/504,753, filed on May 11, 2017.

(51) Int. Cl. A01K 67/027; C07K 16/28; A61K 49/00

(52) U.S. Cl. CPC A01K 67/0278; C07K 16/2866; A61K 49/0008; A01K 2227/105; A01K 2267/0387; C07K 2317/14; A01K 2207/12; A01K 2207/10; A01K 2207/15

(57) ABSTRACT

The present invention provides for inhibition or blockade of immunomodulatory cell receptors to facilitate improved or complete reconstitution of a human immune system in laboratory animals, improve animal health, and improve animal longevity. Thus, the invention relates generally to compositions and methods of generating and using transgenic non-human animals that are engrafted with a human hematopoietic system involving anti-CCR5 agents. In various embodiments, the human hematopoietic system engrafted transgenic non-human animals of the invention are useful as systems for the in vivo evaluation of the growth and differentiation of hematopoietic and immune cells, immune responses, vaccines and vaccination regimens, and human pathogens and production and collection of immune mediators, including human antibodies.

### BACKGROUND

Mice rendered genetically suitable to support human cells and tissues have become a favorite model bridging the gap between mouse models and studies in humans (2009, Legrand et al., Cell Host Microbe 6:5-9; 2007, Shultz et al., Nat Rev Immunol 7:118-130; 2007, Manz, Immunity 26:537-541). Particularly, mice that reconstitute a functional human immune system after engraftment of hematopoietic stem and progenitor cells (HSPCs) are of high interest to study vaccine candidates and the biology of pathogens restricted to humans in vivo, as well as immune function generally.

To achieve efficient xenotransplantation, mice lacking an adaptive immune system and natural killer (NK) cells have been successfully developed in the last years and the major models differ mainly in the background strains used. The first one employs the BALB/c Rag2⁻/⁻yc^{-/-} (DKO) mice, and neonatal intrahepatic HSPC transfer (2004, Traggiai et al., Science 304:104-107; 2004, Gimeno et al., Blood 104:3886-3893). A second model reconstitutes instead NOD/scid/yc−/− (NSG) mice by i.v. or intrahepatic injection of human HSPCs (2002, Ito et al., Blood 100:3175-3182; 2005, Ishikawa et al., Blood 106:1565-1573; 2005, Shultz et al., J Immunol 174:6477-6489). After transfer into these mice, human HSPCs can develop into most of the hematopoietic lineages and the human chimerism is maintained for several months (2004, Traggiai et al., Science 304:104-107; 2005, Ishikawa et al., Blood 106:1565-1573). Overall the composition of engrafted cells is similar in these models but higher human engraftment levels were obtained in NOD-based models (2010, Brehm et al., Clin Immunol 135:84-98).

Humanized mouse model utility has been enhanced by the development of new stocks of immunodeficient hosts, and mouse strains such as NOD-scid IL2ry null mice that lack the IL-2 receptor common gamma chain. These stocks of mice lack adaptive immune function, display multiple defects in innate immunity, and support heightened levels of human hematolymphoid engraftment (Pearson et. al., Creation of “Humanized” Mice to Study Human Immunity, CURR. PROTOC. IMMUNOL. 2008 May; Chapter: Unit—15.21, doi: 10.1002/0471142735.im1521s81). That is, the NOD-scid IL2ry null strain (NSG) which lacks T- B- and NK-cells, permits acceptance of human tissues and are easily engrafted by human peripheral blood (PB) or bone marrow (BM) derived cells (Shultz L D, Ishikawa F, Greiner D L (2007) Humanized mice in translational biomedical research, NAT REV IMMUNOL 7: 118-130; Shultz L D, Brehm M A, Bavari S, Greiner D L (2011) Humanized mice as a preclinical tool for infectious disease and biomedical research. ALM NY ACAD SCI 1245: 50-54).

As severely immunocompromised mice lacking T cells, B cells, and NK cells have become widely used hosts for the xenotransplantation of human cells due to their diminished rejection of cells and tissues of human origin, efforts have continued to improve mouse strains, models, and related methodologies to better simulate human immune function (2004, Traggiai et al., Science 304:104-107; 2002, Ito et al., Blood 100:3175-3182; 2005, Ishikawa et al., Blood 106:1565-1573; 2005, Shultz et al., J Immunol 174:6477-6489).

For example, several approaches have been used to improve human cell engraftment and the potential for unbalanced lineage differentiation in CD34+ cell engrafted mice. These include transient approaches such as hydrodynamic injection of plasmid DNA (2009, Chen et al., Proc Natl Acad Sci USA 106:21783-21788), injections of cytokines, and infections of mice or CD34+ cells with lentiviruses (2010, O'Connell et al., PLoS ONE 5:e12009; 2009, Huntington et al., J Exp Med 206:25-34; 2009, van Lent et al., J Immunol 183:7645-7655). Alternatively, transgenic expression of human MHC molecules has been demonstrated to improve the development of antigen-specific immune responses in vivo (2009, Jaiswal et al., PLoS ONE 4:e7251; 2009, Strowig et al., J Exp Med 206:1423-1434; 2011, Danner et al., PLoS ONE 6:e19826). Nonetheless, overexpression of cytokines might also have detrimental side effects due to the unphysiological expression such as in mice transgenic for GM-CSF, and IL-3 (2004, Nicolini et al., Leukemia 18:341-347). Alternatively, human growth factors have been provided in vivo by genetically engineering mice to replace the mouse genes with their human counterparts resulting in their expression in the appropriate niche at physiological levels. Indeed, faithful replacement of mouse GM-CSF and IL-3 as well as thrombopoietin (TPO) group is reported to have resulted in improved development of human macrophages in the lung and HSPC and HPC maintenance in the bone marrow, respectively (2011, Rongvaux et al., Proc Natl Acad Sci USA 94:5320-5325; 2011, Willinger et al., Proc Natl Acad Sci USA 108:2390-2395). Notably, in human TPO knockin mice, despite a highly increased engraftment level of stem and progenitor cells in the bone marrow, no changes were observed in the periphery, demonstrating the potential existence of limiting factors in the periphery such as destruction by phagocytes.

Here, the present inventor provides for the use of binding agents for CCR5 cell receptors, which are known to modulate human immune function, to further improve human immune system engraftment, improve overall mouse health (e.g., weight maintenance), and increase mouse longevity, in immunocompromised mouse strains together with related models and methodologies. According to the present invention, engrafted mice are provided with a CCR5 binding agent to facilitate or improve engraftment, to further improve overall mouse health (e.g., weight maintenance), and increase mouse longevity, in immunocompromised mouse strains together with related models and methodologies. The present invention achieves mice with humanized immune systems wherein engraftment levels greater than or equal to about 60%, 65%, 70%, 75%, 80%, 85%, 86%, 87%, 88%, 89%, 90%, 91%, 92%, 93%, 94%, 95%, 96%, 97%, 98%, 99%, or 100% in one or more of mouse bone marrow, mouse spleen, and mouse peripheral blood cells as measured by flow cytometry analysis of engrafted human cells. The present invention achieves mice with humanized immune systems wherein overall mouse health is enhanced relative to mice that do not receive a CCR5 binding agent in terms of, for example, weight maintenance, physical activity, and overall appearance. The present invention achieves mice with humanized immune systems wherein mouse longevity is enhanced relative to mice that do not receive a CCR5 binding agent. Such improved longevity may be due to, for example, the delay, reduction, prevention (partial or complete) of xenogeneic graft-versus-host-disease in the mice.

Thus, the compounds and methods of the present invention provide an animal model that better simulates human immune function and that is better able to, and may completely, support and sustain engraftment with a human hematopoietic system, and wherein the mice exhibit improved health and longevity. The humanized mouse model of the present invention may be advantageously used to study vaccine candidates and the biology of pathogens restricted to humans in vivo, as well as immune function generally.

### BRIEF SUMMARY

The present invention provides for inhibition or blockade of immunomodulatory cell receptors, such as the CCR5 cell receptor, to facilitate improved or complete reconstitution of a human immune system in laboratory animals. The present invention makes possible laboratory animals with reconstituted humanized immune systems that have improved health and longevity relative to laboratory animals that do not receive a CCR5 binding agent. Thus, the invention relates generally to CCR5 binding agents and improved animal models, compositions, and methods of generating and using transgenic non-human animals that are engrafted with a human hematopoietic system.

In various embodiments, the human hematopoietic system engrafted transgenic non-human animals of the invention are useful as systems for the in vivo evaluation of the growth and differentiation of hematopoietic and immune cells, for the in vivo assessment of an immune response, for the in vivo evaluation of vaccines and vaccination regimens, for the in vivo study of human pathogens, for the in vivo production and collection of immune mediators, including human antibodies, and for use in testing the effect of agents that modulate hematopoietic and immune cell function.

A preferred embodiment of the present invention provides for an immunocompromised mouse strain provided with xenogeneic hematopoietic stem cell transplantation and an anti-CCR5 cell receptor binding agent that exhibits an improved or fully humanized immune system, improved health and longevity, and methods of making or using such a strain.

### BRIEF DESCRIPTION OF THE SEVERAL VIEWS OF THE DRAWINGS

FIG. 1 shows the effect of PRO 140 on the mean weight in grams of eight (8) xeno-GVHD in NSG mice administered 2 mg PRO 140 intraperitoneally twice a week (starting on day 1). On day (−1) male NSG mice received 2.25 cGy total body irradiation. On day 0 mice received 10⁷ fresh Ficoll-Hypaque-purified normal human bone marrow cells (56 year old male donor) i.v. via tail vein. Control mice received normal human IgG.

FIG. 2 shows the effect of PRO 140 on the survival of eight (8) xeno-GVHD in NSG mice administered 2 mg PRO 140 intraperitoneally twice a week (starting on day 1). On day(−1) male NSG mice received 2.25 cGy total body irradiation. On day 0 mice received 10⁷ fresh Ficoll-Hypaque-purified normal human bone marrow cells (56 year old male donor) i.v. via tail vein. Control mice received normal human IgG. Percentage (%) survival was analyzed by the Kaplan-Meier method and Mantel-Cox log-rank test.

FIG. 3 shows the effect of PRO 140 on the mean weight in grams of eight (8) xeno-GVHD in NSG mice administered 0.2 mg PRO 140 intraperitoneally twice a week (starting on day 1). On day (−1) male NSG mice received 2.25 cGy total body irradiation. On day 0 mice received 10⁷ fresh Ficoll-Hypaque-purified normal human bone marrow cells i.v. via tail vein. Control mice received normal human IgG.

FIG. 4 shows the effect of PRO 140 on the survival of eight (8) xeno-GVHD in NSG mice administered 0.2 mg PRO 140 intraperitoneally twice a week (starting on day 1). On day(−1) male NSG mice received 2.25 cGy total body irradiation. On day 0 mice received 10⁷ fresh Ficoll-Hypaque-purified normal human bone marrow cells i.v. via tail vein. Control mice received normal human IgG. Percentage (%) survival was analyzed by the Kaplan-Meier method and Mantel-Cox log-rank test.

FIGS. 5A, 5B, 5C, and 5D show the effect of PRO 140 on xeno-GvHD in NSG mice. Flow cytometry analysis of engrafted human cells in peripheral blood from PRO 140 dosed i.p. twice/week started on day 1. Peripheral blood (100 uL) was drawn on the days indicated from the saphenous vein into heparinized tubes. There were 8 animals per group and the experiments were performed twice. The left panels represent the high dose (2.0 mg) experiment (FIG. 5A and FIG. 5C) and the right panels represent the low dose (0.2 mg) experiment (FIG. 5B and FIG. 5D).

FIG. 6 shows the effect of PRO 140 on xeno-GVHD in eight (8) NSG mice administered 2 mg PRO 140 intraperitoneally twice a week (starting on day 1). The graph provides a flow cytometry analysis of engrafted human cells in peripheral blood and in bone marrow on day 54. Peripheral blood (100 uL) was drawn on day 54 from the saphenous vein into heparinized tubes. Human antibodies were used to detect CD45+ cells (all differentiated hematopoietic cells). Peripheral blood (PB) and spleen (SPL) p<0.05, BM N.S. There were 8 animals per group (two experimental groups, control IgG and PRO 140). The top three panels are representative of a single mouse from the control IgG group. The bottom three panels are representative of a single mouse from the PRO 140 group. Asterisks next to the absolute cell counts indicate P<0.05 between experimental groups of eight mice.

FIG. 7 shows the engraftment of human bone marrow (BM) in NSG mice. Human antibodies detect CD45+ cells (all differentiated hematopoietic cells, PE-Cy7 fluorochrome) and CD3 (mature T cells, FITC). The image provides a flow cytometry analysis of gated white blood cells in human donor and murine recipient cells before engraftment (top panels, left is donor bone marrow and right is recipient peripheral blood) and a representative murine recipient of PRO 140 high dose experiment at time of euthanasia, day 75 (bottom panels left peripheral blood and right, bone marrow).

FIGS. 8A and 8B show the effect of PRO 140 on the % of human CD4+ cells and xeno-GVHD in eight (8) NSG mice administered 2 mg PRO 140 intraperitoneally twice a week (starting on day 1) (FIG. 8A) and in eight (8) NSG mice administered 0.2 mg PRO 140 intraperitoneally twice a week (starting on day 1) (FIG. 8B). The graph provides a flow cytometry analysis of engrafted human cells in peripheral blood, spleen, and bone marrow at the time of euthanasia.

### DETAILED DESCRIPTION

The invention relates generally to compositions and methods of generating and using transgenic non-human animals engrafted with a human hematopoietic system that involve anti-CCR5 cell receptor binding agents to improve engraftment, animal health, and animal longevity.

### 1. The CCR5 Cell Receptor

The CCR5 cell receptor, or CCR5 receptor, is important in many immune responses. It is likely that CCR5 plays a role in inflammatory responses to infection, although its exact role in normal immune function is not completely defined.

The CCR5 receptor is a C-C chemokine G-coupled protein receptor expressed on lymphocytes (e.g., NK cells, B cells), monocytes, macrophages, dendritic cells, a subset of T cells, etc. CCR5 is predominantly expressed on T cells, macrophages, dendritic cells, eosinophils and microglia. The CCR5 protein belongs to the beta chemokine receptor family of integral membrane proteins. CCR5-chemokine (C-C motif) receptor 5 (gene/pseudogene) (Genetics Home Reference (“CCR5-chemokine”); Samson M, Labbe O, Mollereau C, Vassart G, Parmentier M (1996), Molecular cloning and functional expression of a new human CC-chemokine receptor gene, BIOCHEMISTRY 35: 3362-3367).

The CCR5 receptor spans the cellular plasma membrane seven times in a serpentine manner. The extracellular portions represent potential targets for antibodies targeting CCR5, and comprise an amino-terminal domain (Nt) and three extracellular loops (ECL1, ECL2, and ECL3). The extracellular portions of CCR5 comprise just 90 amino acids distributed over four domains. The largest of these domains are at the Nt and ECL2 at approximately 30 amino acids each (Olson et al., CCR5 Monoclonal Antibodies for HIV-1 Therapy, CURR. OPIN. HIV AIDS, March, 4(2): 104-111 (2009)). Regions of this protein are crucial for chemokine ligand binding, functional response of the receptor, and also HIV co-receptor activity (Barmania F, Pepper M S (2013), C-C CHEMOKINE RECEPTOR TYPE FIVE (CCRS): AN EMERGING TARGET FOR THE CONTROL OF HIV INFECTION, APPLIED & TRANSLATIONAL GENOMICS 2: 3-16).

Chemokines bind to receptors expressed on many cell types, including, for example, leukocytes, endothelial cells, fibroblasts, epithelial, smooth muscle, and parenchymal cells. Chemokines play an important role in leukocyte biology, by controlling cell recruitment and activation in basal and in inflammatory circumstances. In addition, because chemokine receptors are expressed on other cell types, chemokines have multiple other roles, including angiogenesis, tissue and vascular remodeling, pathogen elimination, antigen presentation, leukocyte activation and survival, chronic inflammation, tissue repair/healing, fibrosis, embryogenesis, tumorigenesis, etc.

The CCR5 receptor's cognate ligands include CCL5 (RANTES), CCL3, CCL4 (also known as MIP 1 a and 1/1, respectively), and CCL3L1 (Struyf S, Menten P, Lenaerts J P, Put W, D'Haese A, De Clercq E, et al. (2001), Diverging binding capacities of natural LD78beta isoforms of macrophage inflammatory protein-Ialpha to the CC chemokine receptors 1, 3, and 5 affect their anti-HIV-1 activity and chemotactic potencies for neutrophils and eosinophils, EUROPEAN JOURNAL OF IMMUNOLOGY 31: 2170-2178; Miyakawa T, Obaru K, Maeda K, Harada S, Mitsuya H (2002), Identification of amino acid residues critical for LD78beta, a variant of human macrophage inflammatory protein-I alpha, binding to CCR5 and inhibition of RS human immunodeficiency virus type 1 replication, THE JOURNAL OF BIOLOGICAL CHEMISTRY 227: 4649-4655). CCL5, or RANTES, is a chemotactic cytokine protein. Struyf; Slimani H, Charnaux N, Mbemba E, Saffar L, Vassay R, Vita C, et al. (2003), Interaction of RANTES with syndecan-1 and syndecan-4 expressed by human primary macrophages, BIOCHEMICA ET BIOPHYSICA ACTA 1617: 80-88 (“Slimani”); Barmania F, Pepper M S (2013), C-C CHEMOKINE RECEPTOR TYPE FIVE (CCRS): AN EMERGING TARGET FOR THE CONTROL OF HIV INFECTION, APPLIED & TRANSLATIONAL GENOMICS 2: 3-16 (“Barmania”).

The formation of the CCL5 ligand and CCR5 receptor complex causes a conformational change in the receptor that activates the subunits of the G-protein, inducing signaling and leading to changed levels of cyclic AMP (cAMP), inositol triphosphate, intracellular calcium, and tyrosine kinase activation. These signaling events cause cell polarization and translocation of the transcription factor NF-kB, which results in the increase of phagocytic ability, cell survival, and transcription of proinflammatory genes. Once G-protein dependent signaling occurs, the CCL5/CCR5 receptor complex is internalized via endocytosis.

A complete complex structure of CCL5 in complex with CCR5 has been computationally derived. It is reported that the 1-15 residue moiety of CCL5 is inserted into the CCR5 binding pocket; the 1-6 N-terminal domain of CCL5 is buried within the transmembrane region of CCR5; and the 7-15 residue moiety of CCL5 is predominantly encompassed by the N-terminal domain and extracellular loops of CCR5. CCL5 residues Ala16 and Arg17 and additional residues of the 24-50 residue moiety interact with the upper N-terminal domain and extracellular loop interface of CCR5. It is further reported that the integrity of the amino terminus of CCL5 is crucial to receptor binding and cellular activation. Further, it has been reported that CCL5 and HIV-1 primarily interact with mostly the same CCR5 residues, and share the same chemokine receptor binding pocket (See Tamamis et al., Elucidating a Key Anti-HIV-1 and Cancer-Associated Axis: The Structure of CCL5 (Rantes) in Complex with CCR5, SCIENTIFIC REPORTS, 4:5447 (2014)). It is also separately reported that chemokines, such as the CCL5 ligand, principally bind the CCR5 receptor through ECL2 (Olson et al., CCR5 Monoclonal Antibodies for HIV-1 Therapy, CURR. OPIN., HIV AIDS, March, 4(2): 104-111 (2009)).

### 2. Non-Chemokine CCR5 Cell Receptor Binding Agents

CCR5's exact role in normal immune function is not completely defined. But CCR5 seems to have a broad effect on this process as it has been described to mediate the recruitment of effector T cells, as well as Tregs, to many different target organs (Boieri et al., The Role of Animal Models in the Study of Hematopoietic Stem Cell Transplantation and GvHD: A Historical Overview, FRONTIERS IN IMMUNOLOGY, August 2016 7:333). Accordingly, blocking chemokine-chemokine receptor interaction is a therapeutic strategy that has been tested using animal models. Administration of anti-CXCR3 or anti-CX3CL1 antibodies in mouse models of aGvHD were shown to reduce gastrointestinal aGvHD. However, targeting CCR5 has given contrasting results as this chemokine is thought to be involved also in Treg recruitment to peripheral tissues (ld.).

Various compounds exist that inhibit, interrupt, block, alter, or modify the CCR5/CCL5 receptor/ligand axis (i.e., CCR5 receptor/CCL5 ligand axis). Many of these compounds have been developed for the treatment of HIV-1, which also binds with the CCR5 receptor and is known to share some binding commonalities with CCL5. Such compounds include extracellular or cell transmembrane CCR5 binding agents such as, for example, PRO 140 (extracellular) and maraviroc (transmembrane), and other compounds such as vicriviroc, aplaviroc, SCH-C, and TAK-779, and antibodies such as PA14, 2D7, RoAb13, RoAb14, 45523, etc.

Inhibition of CCR5 signaling has also been shown to have immunosuppressive effects. For example, in vitro studies have been conducted to investigate the effects of CCR5 receptor blockade by maraviroc on activated human T cells on potential immunological mechanisms. It was found that blocking CCR5 by maraviroc not only can block CCR5 and CCR2 internalization processes induced by CCL5 and CCL2, but can also inhibit T-cell chemotactic activities toward their cognate ligands, respectively. Further, blocking CCR5 with maraviroc at high doses tends to decrease production of TNF-α and IFN-γ. It was also noted that the effect of maraviroc on CCR5 was temporary and reversible (Yuan et al., In Vitro Immunological Effects of Blocking CCR5 on T Cells, INFLAMMATION, 38(2): 902-910 (2015); see Arberas et al., In vitro effects of the CCR5 inhibitor maraviroc on human T cell function, J. ANTIMICROB. CHEMOTHER., 68(3): 577-586 (2013)).

It has also been found that the most potently antiviral anti-CCR5 monoclonal antibodies including, for example, PRO 140, bind CCR5 receptor amino acid residues in EL2 alone or in combination with Nt residues. It has also been determined that the CCR5 receptor binding sites for anti-CCR5 monoclonal antibodies are distinct from those of small-molecule CCR5 antagonists. That is, available small-molecule CCR5 antagonists, such as maraviroc, bind the hydrophobic cavity formed by the transmembrane helices, i.e., not the extracellular Nt or loop regions. The amino acid residue E283 in the seventh transmembrane region has been specifically identified as a principle site of interaction for small molecules, and maraviroc and vicriviroc have been found to bind to identical sets of CCR5 receptor amino acids (Olson et al., CCR5 Monoclonal Antibodies for HIV-1 Therapy, CURR. OPIN. HIV AIDS, March, 4(2): 104-111 (2009)). It has also been reported, however, that the CCL5 ligand and maraviroc dock on the CCR5 receptor by sharing two receptor sites: the Nt and the ECL2, and that synthetic CCL5-derived peptides may also be used to block the CCR5 receptor (Secchi et al., Combination of the CCL5-Derived Peptide R4.0 with Different HIV-1 Blockers Reveals Wide Target Compatibility and Synergic Cobinding to CCR5, ANTIMICROB AGENTS CHEMOTHER., 58(10): 6215-6223 (2014)).

PRO 140 binds to the CCR5 receptor and was developed as an entry inhibitor for HIV, has completed seven clinical trials as an investigative HIV therapeutic entity, and is currently in two FDA approved Phase 2b/3 clinical trials for HIV infection. Specifically, PRO 140 is a competitive inhibitor of CCR5 with binding reactivity to the second external loop of CCR5 (Olson W C, Rabut G E E, Nagashima K A, Tran D N H, Anselma D J, Monard S P, et al. (1999), Differential inhibition of human immunodeficiency virus type 1 fusion, gp120 binding, and CC-chemokine activity by monoclonal antibodies to CCRS, JOURNAL OF VIROLOGY 73: 4145-4155 (“Olson”)). Importantly, PRO 140 binding to CCR5 does not result in CCL5 ligand (RANTES) agonist activity, and may dampen such activity, as assessed by downstream triggering of cAMP or tyrosine kinase activity, but does not appear to dampen certain other downstream effects resulting from CCR5 exposure to RANTES (See PCT/US2016/039016).

In one embodiment, the present disclosure provides for the use of a PRO 140 antibody, or binding fragment thereof. PRO 140 is a humanized monoclonal antibody described in U.S. Pat. Nos. 7,122,185 and 8,821,877, which are incorporated herein by reference, in their entirety. PRO 140 is a humanized version of the murine mAb, PA14, which was generated against CD4⁺ CCR5⁺ cells. Olson et al., Differential Inhibition of Human Immunodeficiency Virus Type 1 Fusion, gp 120 Binding and CC-Chemokine Activity of Monoclonal Antibodies to CCR5, J. VIROL., 73: 4145-4155. (1999). PRO 140 binds to CCR5 expressed on the surface of a cell, and potently inhibits HIV-1 entry and replication at concentrations that do not affect CCR5 chemokine receptor activity in vitro and in the hu-PBL-SCID mouse model of HIV-1 infection (Olson et al., Differential Inhibition of Human Immunodeficiency Virus Type 1 Fusion, gp 120 Binding and CC-Chemokine Activity of Monoclonal Antibodies to CCR5, J. VIROL., 73: 4145-4155. (1999); Trkola et al., Potent, Broad-Spectrum Inhibition of Human Immunodeficiency Virus Type 1 by the CCR5 Monoclonal Antibody PRO 140, J. VIROL., 75: 579-588 (2001)).

Nucleic acids encoding heavy and light chains of the humanized PRO 140 antibody have been deposited with the ATCC. Specifically, the plasmids designated pVK-HuPRO140, pVg4-HuPRO140 (mut B+D+I) and pVg4-HuPRO140 HG2, respectively, were deposited pursuant to, and in satisfaction of, the requirements of the Budapest Treaty with the ATCC, Manassas, Va., U.S.A. 20108, on Feb. 22, 2002, under ATCC Accession Nos. PTA 4097, PTA 4099, and PTA 4098, respectively.

In a one embodiment, the methods disclosed herein comprise administering a humanized antibody designated PRO 140 or an antibody that competes with PRO 140 for binding to the CCR5 receptor, wherein the PRO 140 comprises (i) two light chains, each light chain comprising the expression product of the plasmid designated pVK:HuPRO140-VK (ATCC Deposit Designation PTA-4097), and (ii) two heavy chains, each heavy chain comprising the expression product of either the plasmid designated pVg4:HuPRO140 HG2-VH (ATCC Deposit Designation PTA-4098) or the plasmid designated pVg4:HuPRO140 (mut B+D+I)-VH (ATCC Deposit Designation PTA-4099). In a further embodiment, the PRO 140 is a humanized or human antibody that binds to the same epitope as that to which antibody PRO 140 binds. In another embodiment, the monoclonal antibody is the humanized antibody designated PRO 140.

CCR5 is a protein on the surface of white blood cells that functions as a receptor for chemokines. As such, it is an important component in most immune responses. In this manner, T cells are attracted to specific tissue and organ targets. The CCR5 protein belongs to the beta chemokine receptor family of integral membrane proteins. It is a G protein-coupled receptor which functions as a chemokine receptor in the C-C chemokine_group.

CCR5's cognate ligands include CCL3, CCL4 (also known as MIP 1α and 1β, respectively), and CCL3L1. CCR5 also interacts with CCL5, a chemotactic cytokine₋protein also referred to as RANTES.

CCR5 is predominantly expressed on T cells, macrophages, dendritic cells, eosinophils and microglia. It is likely that CCR5 plays a role in inflammatory responses to infection, though its exact role in normal immune function is not completely defined. Regions of this protein are also crucial for chemokine ligand binding, functional response of the receptor, and HIV co-receptor activity.¹⁷

PRO 140 was developed as an entry inhibitor for HIV and has completed seven human clinical trials to determine its therapeutic impact on HIV infection and is currently in two FDA approved Phase 2b/3 clinical trials in HIV patients. It is also being evaluated in a Phase 2 clinical trial for acute GvHD in acute myelogenous leukemia (AML) and myelodysplastic syndrome (MDS) patients undergoing HSCT.

PRO 140 is a competitive inhibitor of CCR5 with binding reactivity to the second external loop of CCR5. Importantly, PRO 140 binding to CCR5 does not result in agonist activity as assessed by downstream triggering of cAMP or tyrosine kinase activity. This characteristic distinguishes PRO 140 from MVR, a small molecule CCR5 inhibitor with agonist activity which is an allosteric antagonist that prevents CCL3, CCL4, and CCL5 ligand signaling.

PRO 140 is an IgG₄ fully humanized monoclonal antibody that was developed as an entry inhibitor for HIV. It binds to the second external loop of CCR5¹⁸ and is a competitive inhibitor of HIV binding to CCR5. PRO 140 binds to CCR5-expressing CD4+ T cells, CD8+ T cells, T regulatory cells, NK cells, NKT cells, and monocytes from human peripheral blood as determined by flow cytometric analysis. PRO 140 binding to CCR5 does not trigger agonist activity as assessed by downstream activation of cAMP or tyrosine kinase activity.

3. Production of Transgenic Non-Human Animals that are Engrafted with a Human Hematopoietic System

Hematopoietic stem cells may be sourced from, for example, bone marrow, peripheral blood, and cord blood.

Generally, two basic protocols describe generating humanized mice: Basic Protocol 1 deals with hematopoietic stem cell (HSC) engraftment (human SCID repopulating cell; hu-SRC) and Basic Protocol 2 addresses engraftment with human peripheral blood mononuclear cells (PBMC) (human peripheral blood leukocyte; hu-PBL) (Pearson et. al., Creation of “Humanized” Mice to Study Human Immunity, CURR. PROTOC. IMMUNOL. 2008 May; Chapter: Unit—15.21, doi: 10.1002/0471142735.im1521s81).

The main advantage of the HSC engraftment model (hu-SRC-SCID) is that the human T and B cells develop from human stem cells engrafted in the mouse, undergo negative selection during differentiation into T and B cells, and are therefore tolerant of the mouse host. This model allows for investigation of hematopoietic lineage development and mechanisms of immune system development and the generation of primary immune responses by a naive immune system (Pearson et. al., Creation of “Humanized” Mice to Study Human Immunity, CURR. PROTOC. IMMUNOL. 2008 May; Chapter: Unit—15.21, doi: 10.1002/0471142735.im1521s81).

The PBMC model (hu-PBL-SCID) utilizes leukocytes isolated from peripheral whole blood or spleen and allows for rapid analysis of human immune function because the transferred lymphocytes are functionally mature. This model is best suited for studies of immune function from patients with immunologic disorders, analyses of antigen recall responses, investigations of allograft rejection, and other short-term (-4-week) experiments (Pearson et. al., Creation of “Humanized” Mice to Study Human Immunity, CURR. PROTOC. IMMUNOL. 2008 May; Chapter: Unit—15.21, doi: 10.1002/0471142735.im1521s81).

In many instances, total body irradiation (TBI) prior to engraftment has been a standard conditioning regimen to achieve high levels of human cell engraftment in xenograft animal models because it triggers the secretion of stem cell factor (SCF), which is critical for hematopoietic stem cell engraftment, proliferation, and survival. However, other conditioning regimens, including depletion of mouse macrophages or granulocytes prior to engraftment or administration of chemotherapeutic agents, such as bulsulfan, have been explored (Kang et al., Humanizing NOD/SCID/IL-2Rynull (NSG) mice using busulfan and retro-orbital injection of umbilical cord blood-derived CD34⁺ cells, BLOOD RESEARCH, 2016 March; 51(1): 31-36; Pearson, 2008). Additional conditioning regimen efforts to improve engraftment have included, for example, treatment of engrafted mice with human cytokines, or co-engraftment with mesenchymal stem cells (Pearson, 2008).

The present invention focuses on the HSC engraftment model for the generation of chimeras by xeno-transplantation. Accordingly, this model encompasses the administration of human cells or tissue in usually immunodeficient animals. Excellent host animals for generating a human immune system are mouse lines that have several defects in the adaptive immunity such as Rag^{2-/-/}y^{-/-}, BNX or NOD/SCID B2m^{null}.

In a preferred embodiment, the present invention uses NOD. Cg-Prkdc^{scid}Il2ry^{tm1Wjl}/SzJ (NOD-scid IL2ry^{null}, NSG) mice.

Different lines of the NOD/SCID (non-obese-diabetic/severe combined immunodeficiency) mouse serve as a standard model for humanization. They are characterized essentially by the following immunodeficiency properties: complete loss of B lymphocytes and T lymphocytes, reduced number of NK cells, defects in the differentiation and function of antigen-presenting cells and the absence of circulating complement. These mice are more susceptible for ionizing radiation than the wild type and have defects in the DNA repair system. The formation of human individual lines or several lines of hematopoiesis in an immunodeficient animal is possible after transplantation of human hematopoietic stem cells, differentiated hematopoietic cells as well as lymphoid organs.

Here, the present inventor found that administration of an anti-CCR5 binding agent to immunodeficient mice provided improved engraftment, in terms of engraftment success, animal health, and animal longevity, using the HSC engraftment model for the generation of chimeras by xeno-transplantation. Specifically, a humanized monoclonal antibody, PRO 140, was administered to NOD-scid IL2ry^{null}, NSG mice upon HSC engraftment. Surprisingly, mice administered PRO 140 exhibited significantly improved health (e.g., weight maintenance and appearance), and longevity (e.g., 100% survival past 70 days in a xeno-transplant animal model), while also demonstrating successful engraftment.

4. Animal Model Studies Including Transgenic Non-Human Animals Engrafted with a Human Hematopoietic System

Graft-versus-host disease (GvHD) is an exemplary human disease for study by a transgenic non-human animal (here, mouse) engrafted with a human hematopoietic system. Alteration of mouse models used to study GvHD continue to offer insights into the extreme complexities of human immune function generally and, specifically, in this disease pathology.

As new therapeutic options to treat GvHD are badly needed, the humanized mouse models used to study this disease, and related modifications to these models, offer valuable insights into whether and how such modifications to mouse strains, mouse models, and related methodologies may impact humanized mouse immune systems, engraftment, and related human therapeutic options.

GvHD is noted here with particular interest with respect to immune cell trafficking and the inventor's focus on modulation of CCR5 cell receptor binding because GvHD pathophysiology includes migration of lymphocytes to their target tissues as one of the key steps. That is, it is understood that chemokines and chemokine receptors, such as the CCR5 cell receptor, specifically guide T cells in this process.

Accordingly, the present invention provides improved non-human animal models and methodologies to address the role of chemokines and chemokine receptors on engraftment of more human immune systems, and maintenance of animal health and longevity, in immunocompromised mice.

### EXAMPLES

These examples describe the present invention as realized in a Graft-versus-Host-Disease (GvHD) mouse model. As stated elsewhere in this application, GvHD is a prevalent and potentially lethal complication following hematopoietic stem cell transplantation. Humanized mouse models of xenogeneic-GvHD are important tools to evaluate the human immune response in vivo.

It is noted that GvHD can develop, for example, following allogeneic hematopoietic stem cell transplantation (HSCT), which has an important role in a variety of malignant and non-malignant hematological diseases. Donor derived T-cell alloreactivity to human leukocyte antigens (HLA) disparities can result in GvHD which is potentially life threatening. New therapies are needed to address GvHD other than lymphoid depletion strategies as this non-specific approach leaves patients at risk of complications such as infection or cancer relapse (Champlin R, Ho W, Gajewski J, Feig S, Burnison M, Holley G, et al. (1990), Selective depletion of CD8+ T lymphocytes for prevention of graft-versus-host disease after allogeneic bone marrow transplantation, BLOOD 76: 418-423; Gallardo D, Garcia-Lopez J, Sureda A, Canals C, Ferra C, Cancelas J A, et al. (1997), Low-dose donor CD8+ cells in the CD4-depleted graft prevent allogeneic marrow graft rejection and severe graft-versus-host disease for chronic myeloid leukemia patients in first chronic phase, BONE MARROW TRANSPLANT 20: 945-952).

For example, GvHD in the Hu-SRC-SCID model for NSG mice is dependent on human immune cell xeno-reactivity against mouse Major Histocompatibility Class I and class II antigens (MHC) similar to HLA mismatched HSCT where donor alloreactivity is initiated by recognition of recipient MHC antigens (King M A, Covassin L, Brehm M A, Racki W, Pearson T, Leif J, et al. (2009) Human peripheral blood leucocyte non-obese diabetic-severe combined immunodeficiency interleukin-2 receptor gamma chain gene mouse model ofxenogeneic graft-versus-host-like disease and the role of host major histocompatibility complex. CLIN EXP IMMUNOL 230 157: 104-118; Reddy P, Ferrara J L (2003) Immunobiology of acute graft-versus-host disease. BLOOD REV 17: 187-194). The involvement of specific organs in acute GvHD of HSCT recipients suggests that immune cell trafficking is crucial to the pathophysiology of this disease.

Here, the inventor evaluated PRO 140, a humanized monoclonal antibody which targets a chemokine receptor, C-C chemokine receptor type 5 (CCR5 or CD195) as an inhibitor of the development of xeno-GvHD. Inhibition of lymphocyte trafficking using a CCR5 antagonist has previously been shown to reduce the impact of acute GvHD in patients undergoing HSCT (Reshef R, Luger S M, Hexner E O, Loren A W, Frey N V, Goldstein S C, et al. (2011), Inhibition of lymphocyte trafficking using a CCR5 antagonist—final result of a phase I/II study, BLOOD 118: 1011; Reshef R, Mangan J K, Luger S M, Loren A W, Hexner E O, Frey N Y, et al. (2014), Extended CCR5 blockade in graft-versus-host disease prophylaxis—a phase II study, BLOOD 124: 2491; and Moy R H, Huffman A P, Richman L P, Crisalli L, Wang X K, Hoxie J A, et al. (2017), Clinical and immunologic impact of CCR5 blockade in graft-versus-host disease prophylaxis, BLOOD 129: 906-916).

As discussed below, administration of PRO 140 to the NSG mice following injection of hematopoietic stem cells resulted in a dramatic, significant, and surprising increase in mouse health and survival, a positive GvHD therapeutic effect, and human CD45+ cell engraftment levels after 70 days of greater than about 75% in peripheral blood and greater than about 65% in bone marrow.

Here NOD-scid IL-2Ry^{null} mice (NSG) were transplanted with human bone marrow cells to evaluate the role of immune cell trafficking in the production of acute GvHD. PRO 140 was used to evaluate its influence on bone marrow cell engraftment and modulation of acute GvHD. Engraftment kinetics were evaluated by assessing human CD45+ cells and CD3+T-cells in treated and control mice. In peripheral blood, spleen and bone marrow, PRO 140 treated mice showed no signs of GvHD throughout the 70-day study period and gained weight until sacrifice at 70 days for flow cytometry analysis. The control mice started losing weight after 25 days, showed classic signs of GvHD (ruffled fur, lethargy, etc.) and all required sacrifice by Day 54. The percentage of human CD45+ cells in peripheral blood increased in both groups of mice throughout the 50-day comparison period but was significantly lower in the PRO 140 treated mice at day 50. Importantly, there was no difference in control and PRO 140 treated mice in human CD45+ cells detected in bone marrow at Day 70. By masking the CCR5 chemokine receptor, PRO 140 eliminated acute GvHD in this humanized mouse model without significantly altering engraftment.

### Animal Studies:

Animal experiments were conducted in accordance with the ethical standards and according to national and international guidelines and were approved by the Cleveland Clinic Institutional Animal Care and Use Committee. Male NSG mice, NOD. Cg-Prkdc^{scid}Il2ry^{tm1Wjl}/SzJ (NOD-scid IL2ry^{null}, NSG) mice were obtained from the Jackson Laboratory (Bar Harbor, Me., USA), and athymic nude (nu/nu) (Taconic, Hudson, N.Y.) 6-8 wk old were used. Mice were housed in a barrier facility in cages with microisolator lids, autoclaved bedding, and HEPA-filtered air, and maintained under 12:12 light/dark cycles, controlled temperature and humidity. Animals had free access to autoclaved standard food and filtered water. Conditioning regimen: Mice received 2.25 Gy total body irradiation via a 137Cs source (Shepherd, Los Angeles Calif.).

Bone Marrow Transplantation and Generation of Xeno-GvHD:

Following gamma irradiation (24 h later) mice were engrafted with human BM cells. De-identified human donor cells were obtained by back-flushing filter packs utilized by the Cleveland Clinic BMT program. Fresh (non-frozen) leukocytes were purified by Ficoll-Hypaque gradient centrifugation, washed in phosphate buffered saline (PBS), assessed for viability (ViCell, Beckman Coulter, Brea, Calif.). Human BM leukocytes were injected into the lateral tail vein (107 cells/mouse). Mice were monitored for clinical symptoms of GvHD (body posture, activity, fur and skin condition, weight loss) two times/wk. Peripheral blood was monitored weekly for engraftment utilizing saphenous vein venipuncture (50 mL) collected in K-EDTA tubes. Mice exhibiting 20% weight loss with clinical symptoms of GvHD were considered to have reached experimental endpoint and subject to euthanasia by controlled gradient CO₂ inhalation.

PRO 140 Treatment:

Mice were randomized into control and treatment groups of 8 animals each by body weight. PRO 140 was administered intraperitoneally (i.p.) at two doses, 2.0 or 0.2 mg/mouse twice weekly. The 2.0 mg dose was calculated^{20,21} to approximate the dose used in an ongoing CytoDyn sponsored phase 2 human clinical trial for acute GvHD. A single administration of this dose in HIV positive patients has been shown to reduce the HIV load by more than ten-fold. The 0.2 mg dose was used as a lower limit of activity as it did not significantly reduce the HIV load in HIV positive patients. Control mice received normal human IgG (Sigma Aldrich, St. Louis, MOPRO 140 dosage was calculated using “Representative Surface Area to Weight Ratios (km) for Various Species” from: Freireich et al., Quantitative comparison of toxicity of anticancer agents in mouse, rat, hamster, dog, monkey, and man, CANCER CHEMOTHER REP., 50:219-44 (1966); and the National Cancer Institute Developmental Therapeutics Program http://dtp.nci.nih.gov. Starting with the human dose of PRO 140=5.8 mg/kg×12 (man-to-mouse conversion factor)=69.6 mg/kg mouse dose; average mouse=0.025 kg, therefore dose is 69.6 mg/kg×0.025 kg=1.74 mg (mouse single dose). This was rounded up to 2.0 mg and designated as the “high dose”. A “low dose” (0.2 mg) was also tested. IgG derived from human serum (>95% SDS-PAGE, Sigma, I4506) was used as a non-specific control antibody.

Flow Cytometry:

Peripheral blood (PB), bone marrow (BM), and spleen (SPL) samples were analyzed by flow cytometry. Splenocytes were passed through a 40 mm strainer. Erythrocytes were lysed with ammonium chloride, cells were washed twice with PBS and stained for 15 min at 4 deg C. in PBS/0.5 mM EDTA/0.5% BSA with the following antibodies: anti-human-CD3-FITC (clone UCHT1, IM1281U), anti-human-CD45-PC7 (clone J.33, IM3548U), anti-mouse-CD45.1-FITC (clone A20), eBioscience (Thermo Fisher) and anti-human-CD56-PE (clone 5.1H11), Biolegend. CountBright beads (Thermo Fisher) were added (50 μL) to samples to determine absolute cell numbers. For human CD45, mouse CD45, and human CD3, results were expressed as percentage of total events, and also as absolute accumulated cell number. For human CD56, results were expressed as percentage of total events, and also as cells/μL peripheral blood. Samples were analyzed on a Cytomics FC500 Flow Analyzer (Beckman/Coulter).

Statistical Analysis:

Statistical analysis was performed using GraphPad Prism (GraphPad Software, La Jolla, Calif.). All measures of variance were depicted as standard error of the mean (SEM). Survival was analyzed by the Kaplan-Meier method and Mantel-Cox log-rank test. For other data, two-sided unpaired Students t-test was used.

### RESULTS

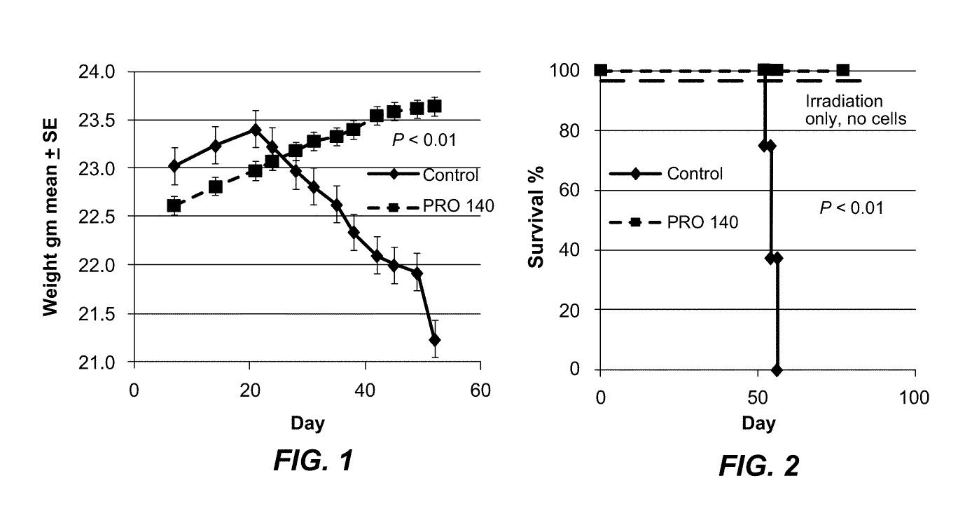

The effects of PRO 140 on the development of acute GvHD was evaluated in the xenogeneic NSG mouse model. Two doses of PRO 140 or control IgG (2 mg and 0.2 mg i.p. twice weekly) were used with the high dose calculated to approximate the dosing used in an ongoing phase 2 clinical trial of acute GvHD. By necessity, the high and low dose studies were done in succession using different BM donors. An assessment of the hallmarks of GvHD in MSG mice including observed physical signs (ruffled fur, lethargy, severe hunching), measured weight loss, and death, were determined in both experiments. In the high dose study, physical signs of GvHD were observed in control mice starting at day 25 after engraftment with BM from a 56-year-old donor and included ruffled fur, lethargy, hunching, and weight loss. Weight loss continued in the control group and was significantly different (P<0.01) from the PRO-treated group which showed no physical signs of GvHD and continued to gain weight (FIG. 1). When survival was assessed in a Kaplan-Meier plot (FIG. 2), the results were highly statistically significant (P<0.01) with all of the control animals dead by 56 days and all of the PRO 140 treated animals alive at 75 days when sacrificed for flow cytometry analysis of engraftment.

FIG. 1. Effect of PRO 140 on xeno-GVHD in NSG mice—weight; high dose.

On day (−1) male NSG mice received 2.25 cGy total body irradiation. On day 0 mice received 107 fresh Ficoll-Hypaque-purified normal human bone marrow cells (56 year old male donor) i.v via tail vein. Control mice received normal human IgG. As can be seen, the control mice starting losing weight at about 20 days after engraftment, and this weight loss continued from a high of about 23.4 gm at about 20 days to about 21.2 gm after about 52 days. Meanwhile the mice in the PRO 140 treated group gained weight over the same time period, going from about 23.0 gm at about 20 days to about 23.6 gm at about 52 days.

FIG. 2. Effect of PRO 140 on xeno-GVHD in NSG mice—survival; high dose.

On day (−1) male NSG mice received 2.25 cGy total body irradiation. On day 0 mice received 107 fresh Ficoll-Hypaque-purified normal human bone marrow cells (56 year old male donor) i.v via tail vein. Control mice received normal human IgG. As shown in FIG. 2, all of the control animals were dead by 56 days and all of the PRO 140 treated animals alive at 70 days when sacrificed for flow cytometry analysis of engraftment.

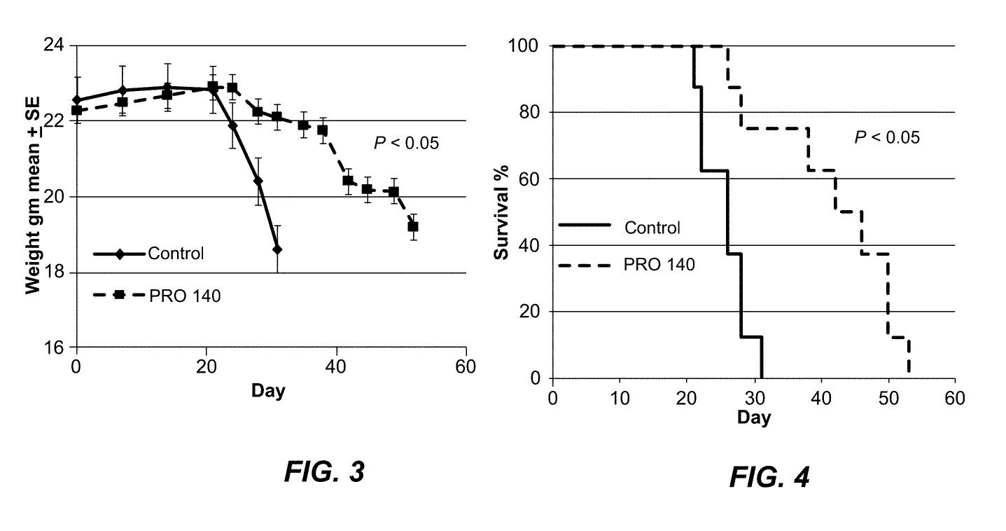

FIG. 3 and FIG. 4 show the effect of PRO 140 on xeno-GVHD in NSG mice at a low 0.2 mg dosing schedule with respect to weight and survivability. On day (−1) male NSG mice received 2.25 cGy total body irradiation. On day 0 mice received 10⁷ fresh Ficoll-Hypaque-purified normal human bone marrow cells (male donor) i.v. via tail vein. Control mice received normal human IgG. On day 1 mice received 0.2 mg PRO 140 i.p. n=8 mice/group.

By necessity, the high and low dose studies were done in succession using different BM donors. In the two sets of experiments, BM donors of different ages were used. Consistent with published data, the younger donor used on the low dose cohort resulted in more aggressive GvHD when time to death was compared (31 days vs. 54 days, FIGS. 2,4) (Rezvani A R, Storer B E, Guthrie K A, et al. (2015) Impact of donor age on outcome after allogeneic hematopoietic cell transplantation, BIOL BLOOD MARROW TRANSPLANT 21(1): 105-112). The extent to which the lower dose of PRO 140 versus a more aggressive BM contributed separately to the weight loss and the Kaplan-Meier plots was not independently assessed in this study.

In the low dose study using one-tenth the dose, physical signs of GvHD were observed in control mice starting at day 20 after engraftment with BM from a 26-year-old donor and included ruffled fur, lethargy and hunching with weight loss starting shortly thereafter. Weight loss continued in the control group and was significantly different (P<0.05) from the PRO 140-treated group which showed physical signs of GvHD and weight loss starting at day 25-28 (FIG. 3). When survival was assessed in a Kaplan-Meier plot (FIG. 4), the results were statistically significant (P<0.05) with all of the control animals dead by 31 days and all of the PRO 140-treated animals dead by 54 days. As indicated by survival time in control animals from high and low dose studies (54 vs. 31 days), the younger BM donor produced more aggressive GvHD.

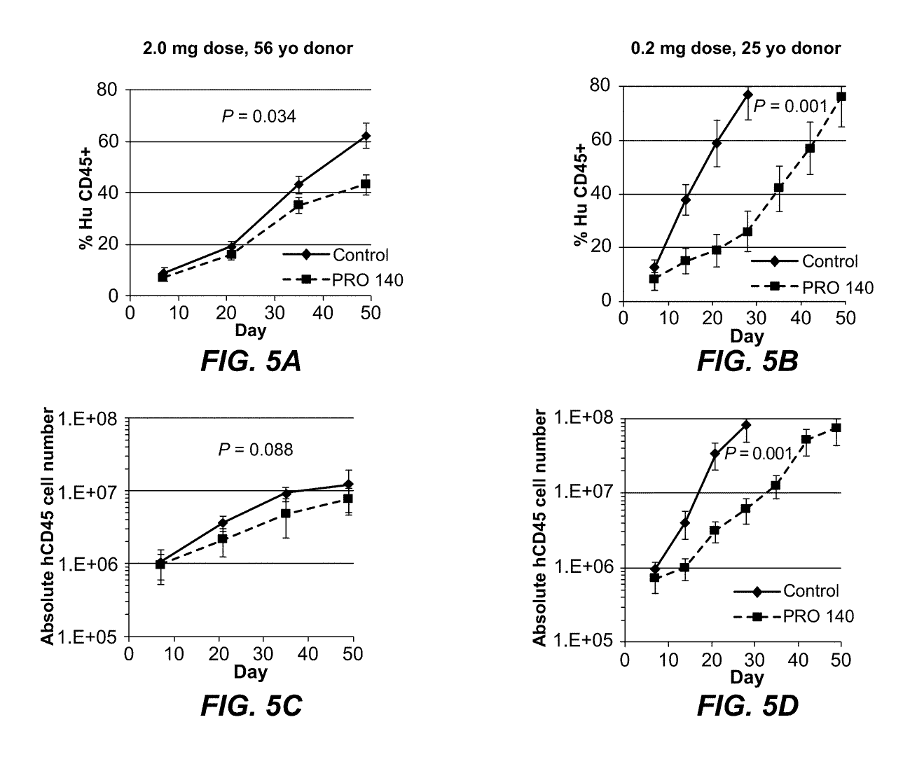

FIGS. 5A, 5B, 5C, and 5D show the effect of PRO 140 on xeno-GvHD in NSG mice. Flow cytometry analysis of engrafted human cells in peripheral blood from PRO 140 dosed i.p. twice/week started on day 1. Peripheral blood (100 uL) was drawn on the days indicated from the saphenous vein into heparinized tubes. There were 8 animals per group and the experiments were performed twice. The left panels represent the high dose (2.0 mg) experiment (FIG. 5A and FIG. 5C) and the right panels represent the low dose (0.2 mg) experiment (FIG. 5B and FIG. 5D).

FIGS. 5A, 5B, 5C, and 5D show the effect of PRO 140 on xeno-GvHD in NSG mice. Flow cytometry analysis of engrafted human cells in peripheral blood from PRO 140 dosed i.p. twice/week started on day 1. Peripheral blood (100 uL) was drawn on the days indicated from the saphenous vein into heparinized tubes. There were 8 animals per group and the experiments were performed twice. The left panels represent the high dose (2.0 mg) experiment (FIG. 5A and FIG. 5C) and the right panels represent the low dose (0.2 mg) experiment (FIG. 5B and FIG. 5D).

Analysis of the kinetics of engraftment in the peripheral circulation by flow cytometry using an antibody specific for human CD45+ cells (all differentiated hematopoietic cells), showed similar engraftment for the first 30 plus days (FIGS. 5A, 5B, 5C, and 5D), then diverged with significantly less cells detected in the CD45+ compartment at 50 days in the PRO 140 animals (62% vs. 43%, p=0.034). This is at a time when the control animals are exhibiting severe GVHD. It is contemplated that the PRO 140 reduced inflammation leading to the lower human CD45+ cells count at 50 days. In the low dose cohort, there was a divergence in engraftment starting at 15 days. The same percentage of CD45+ engraftment was achieved albeit approximately 20 days later in the low dose PRO 140 treated mice (P<0.01). This observation was supported by a determination of the absolute number of cells in the peripheral circulation during this time frame (FIG. 5C and FIG. 5D).

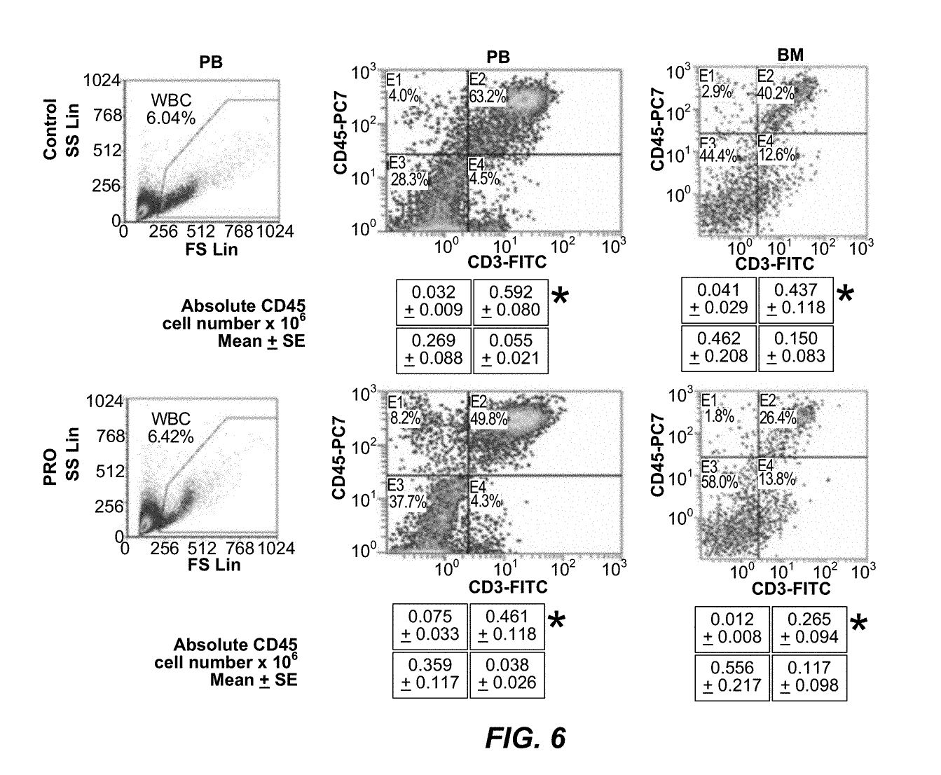

FIG. 6 depicts engraftment of human BM in NSG mice using antibodies to human (hu CD45) and mouse (m CD45) CD45. These antibodies were used to detect the percent of engraftment in bone marrow (BM) and peripheral blood (PB) of PRO 140 treated mice engrafted with human bone marrow cells.

In the high dose cohort at 54 days, analysis of engraftment was assessed in peripheral blood and bone marrow with antibodies specific for CD45 (identifies all differentiated hematopoietic cells) and CD3 (mature T Cells). In PB, the engraftment of mature T-cells was greater in control compared to PRO 140-treated animals (63.2% vs. 49.8%, FIG. 6, PB panels, E2 quadrants). In the BM compartment, control animals exhibited more mature T-cells than the PRO 140 animals (40.2% vs. 26.4%, FIG. 6, BM panels E2 quadrants). This occurred while control animals were experiencing severe GvHD while the PRO 140 animals were gaining weight without signs of GvHD. A determination of the absolute number of cells in each quadrant supported this observation (FIG. 6).

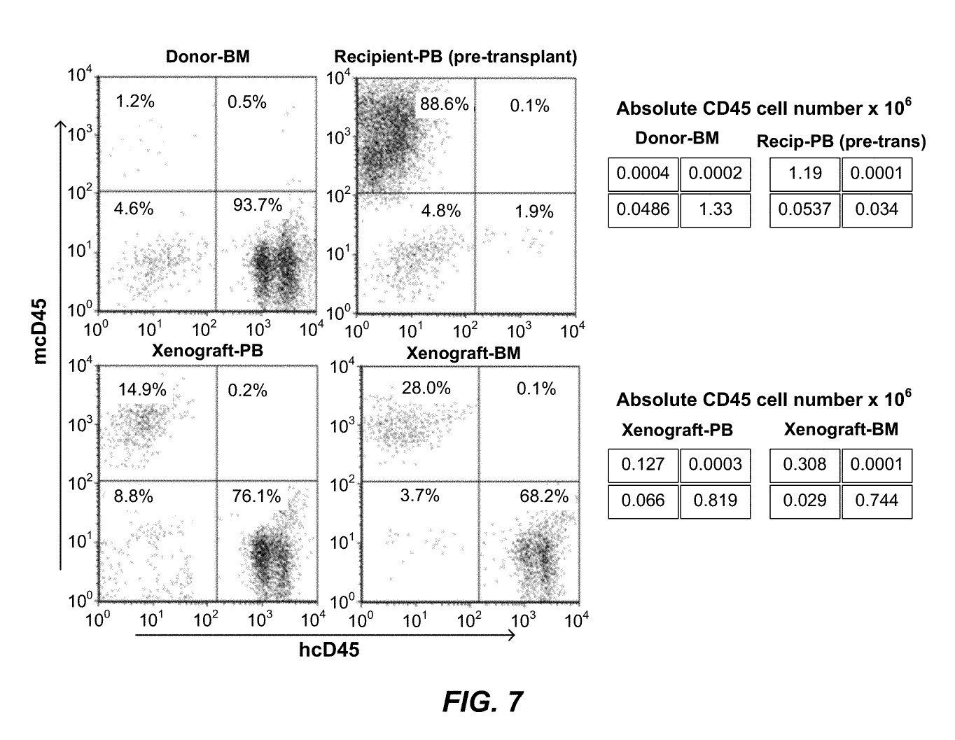

An analysis of engraftment was carried out with PB and BM in the high dose cohort at the time of euthanasia (Day 75) by flow cytometry with antibodies specific for human and mouse CD45. The human donor BM was 93.7% positive for human CD45 and the mouse recipients before engraftment were 88.6% positive for mouse CD45 (FIG. 7, top panels left and right, respectively). Seventy-five days after engraftment, PB in the mice was 76.1% positive for human CD45 and BM was 68.2% positive for human CD45 (FIG. 7, bottom left and right panels, respectively). Mouse hematopoietic cells from PB and BM were 14.9% and 28% respectively. This was consistent with a determination of the absolute number of cells of human or mouse origin (FIG. 7).

It is contemplated that, beyond 70 days, engraftment may continue towards completion, i.e., to achieve mice with humanized immune systems with engraftment levels greater than or equal to about 60%, 65%, 70%, 75%, 80%, 85%, 86%, 87%, 88%, 89%, 90%, 91%, 92%, 93%, 94%, 95%, 96%, 97%, 98%, 99%, or 100% in one or more of mouse bone marrow, mouse spleen, and mouse peripheral blood cells as measured by flow cytometry analysis of engrafted human cells.

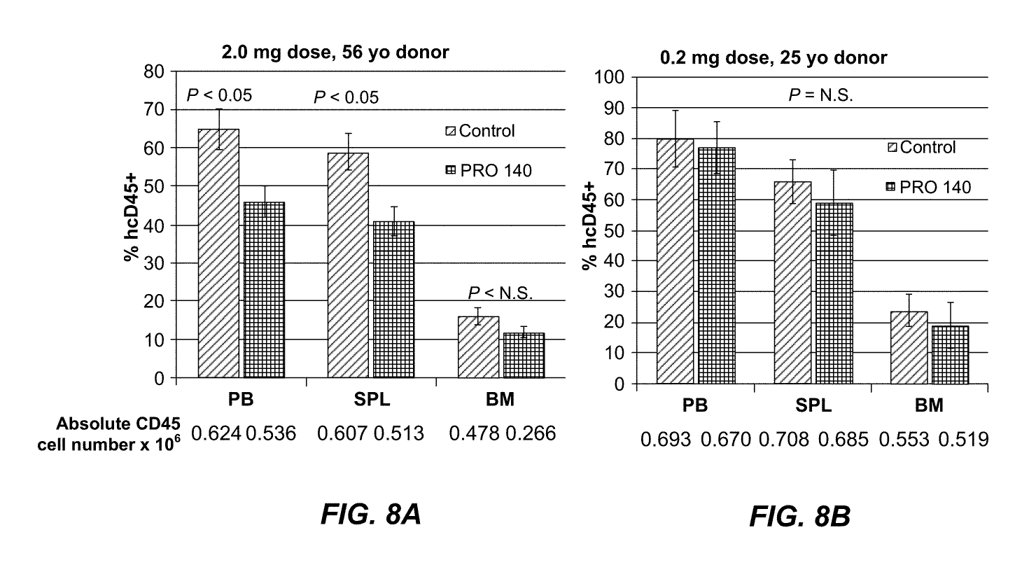

Further analysis of engraftment assessed in peripheral blood and bone marrow at 54 days by flow cytometry with antibodies specific for CD45 (all differentiate hematopoietic cells) and CD3 (mature T Cells) showed more mature T cells in the bone marrow of control animals than of the PRO 140 animals (40.2% vs. 26.4, FIG. 6). The pattern of the flow analysis was also altered in the peripheral blood with the appearance of a new population of cells in the control (GVHD animals, FIG. 6, PRO 140 window E3). It is contemplated that PRO 140 reduced inflammation in the PRO 140 treated animals, leading to lower maturation rates for T cells. At the time of euthanasia, an additional flow analysis was performed with the antibody on peripheral blood, spleen and bone marrow cells (FIG. 8A and FIG. 8B). There was no difference between control and PRO 140 treated animals in the bone marrow, indicating equivalent engraftment. And this indicated that PRO 140 did not inhibit engraftment. Both spleen (58% vs. 41%) and peripheral blood (64% vs. 45%) had significantly more CD45+ cells in control (late stage GVHD) versus Pro 140 treated animals (p<0.05).

This difference was likely attributable to the late stage GvHD ongoing in the control animals at this time. A determination of the absolute number of cells was consistent with these observations (FIG. 8A and FIG. 8B).

### Discussion

The present invention provides for inhibition or blockade of immunomodulatory cell receptors, such as the CCR5 cell receptor, to facilitate improved or complete reconstitution of a human immune system in laboratory animals. The present invention makes possible laboratory animals with substantially or completely reconstituted humanized immune systems that have improved health and longevity relative to laboratory animals that do not receive a CCR5 binding agent. Thus, the invention relates generally to CCR5 binding agents and improved animal models, compositions, and methods of generating and using transgenic non-human animals that are engrafted with a human hematopoietic system.

Here, the inventor exemplifies the present invention using immunodeficient mice with the targeted IL-2Rynull mutation, namely NSG mice, which have been established as a model of choice for engraftment by HSCT for the study of therapeutic approaches for GvHD. This model allowed evaluation of the role of a potent CCR5 inhibitor, PRO 140, on the role of immune cell trafficking in the pathogenesis of GvHD. Importantly, however, it was found that PRO 140 is not only a robust inhibitor of acute GvHD in this model system as measured by physical signs, weight loss, and survival curves, but that overall mouse health and longevity was significantly improved. Thus, the inventors found that treating immunocompromised animals with PRO 140 along with engraftment gives rise to an improved mouse model for studying human immune functionality in healthier, longer-lived mice with substantially, or possibly, completely, reconstituted human immune systems (FIGS. 1 and 2).

The rationale for this approach is based on the role of CCR5, the C-linked protein receptor for CCL5 (aka RANTES), which is a potent chemokine involved in immune cell trafficking. Immune cell trafficking is believed to be crucial for the development of acute GvHD which involves cutaneous and organ involvement including spleen, small intestine and liver, with some involvement of bone marrow and thymus. We did not conduct a histological evaluation of organ involvement in these studies and therefore cannot attribute the effects of PRO 140 to modulation of immune cell trafficking. We plan to do so in follow-on mechanistic studies. Previous murine and human clinical trials have shown that blockade of CCR5 using a small molecule inhibitor, MVR, can reduce the clinical impact of acute GvHD without significantly affecting engraftment. We have previously shown that PRO 140 is a competitive inhibitor of HIV binding to CCR5 without triggering agonist activity, the stimulation of downstream activation markers, or cAMP and tyrosine kinases. These latter characteristics distinguish PRO 140 from MVR.

CCR5 and its natural ligands have also been implicated in transplant organ rejection. Lymphocyte recruitment to tissues involved in GvHD is dependent on CCR5, and migration of CD8+ cells into target organs in murine models is reduced by CCR5 antibody inhibitors resulting in protection against GvHD. CCR5 genetic deletions in mice have resulted in conflicting results in regards to protection from GvHD. In humans, certain CCR5 polymorphisms are protective towards GvHD and correlate with survival in patients with allogeneic bone marrow transplants.

The present study answered an important question regarding the use of PRO 140 in attempts to abrogate GvHD is whether it would have effects on engraftment. It was found to have no such effects in peripheral blood in early stages of engraftment and in peripheral blood and bone marrow in late stages of engraftment. However we did observe significantly more CD45+ cells in animals experiencing severe GvHD than in PRO 140 animals at 50 days. There were also more CD45+ cells in peripheral blood (64% vs. 46%) and spleen (59% vs. 41%) at the time of euthanasia in the GvHD animals (control group) as compared to PRO 140 treated animals with no signs of GvHD. There were no differences in CD45+ cells in bone marrow at this time which suggests that PRO 140 did not negatively affect engraftment. There were also more mature T-cells (CD3+) in the bone marrow of control (GvHD animals) compared to PRO 140 treated animals at Day 54 when the control animals required sacrifice (severe GvHD).

We plan to evaluate the observed functional tolerance on the continued dependence of PRO 140 treatment, clonal deletions, and/or the contributions of regulatory cell activity in addition to a potential role for NK cells and other yet to be defined mechanisms. In future studies we will also assess the functional aspects of the human immune cells in the engrafted mice treated with PRO 140. It should be pointed out that here we used a severely immunocompromised mouse model for the production of xeno-GvHD. We plan to evaluate PRO 140 in allogeneic GvHD mouse models in future experiments. This is an important consideration when bone marrow stem cell transplantation is used in patients with blood cell malignancies such as AML. The graft versus cancer (GVL) response often correlates with the GvHD response. In additional experiments in the future we plan to evaluate the GVL response in animals with reduced or eliminated GvHD responses during PRO 140 treatment.

Taken together these data suggest that the CCR5 receptor on engrafted cells is critical for the development of acute GvHD in this model system and that blocking this receptor from recognizing chemokines in the CCR family is a viable approach to, not only mitigating acute GvHD. Moreover, the significant results achieved by the use of an anti-CCR5 binding agent in engrafted mice in terms of mouse health and longevity, and possibly substantial or complete human hematopoietic engraftment, gives rise to a further improved mouse model for human immune studies. As the NSG model system has been widely accepted as a reliable model for allogeneic GvHD in humans, we believe that PRO 140 has a place in investigative approaches to resolving acute GvHD in AML and MDM patients undergoing stem cell transplantation and, possibly, for use in new mouse models for the study of human immune function.

The various embodiments described above can be combined to provide further embodiments. All of the U.S. patents, U.S. patent application publications, U.S. patent applications, foreign patents, foreign patent applications and non-patent publications referred to in this specification and/or listed in the Application Data Sheet, including U.S. Provisional Patent Application Nos. 62/504,753 and 62/585,974, are incorporated herein by reference, in their entirety. Aspects of the embodiments can be modified, if necessary to employ concepts of the various patents, applications and publications to provide yet further embodiments.

These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

1. A non-human transgenic animal comprising a humanized immune system and an anti-CCR5 cell receptor binding agent.

2. The animal of claim 1, wherein the animal immune system is greater than about 60%, 65%, 70%, 75%, 80%, 85%, 86%, 87%, 88%, 89%, 90%, 91%, 92%, 93%, 94%, 95%, 96%, 97%, 98%, 99%, or is 100% engrafted as evidenced by human CD4+ cell counts in one of peripheral blood or bone marrow.

3. The animal of claim 1, wherein the animal immune system is greater than about 90% engrafted as evidenced by human CD4+ cell counts in one of peripheral blood or bone marrow.

4. The animal of claim 1, wherein the animal immune system is greater than 98% engrafted as evidenced by human CD4+ cell counts in one of peripheral blood or bone marrow.

5. The animal of claim 1, wherein the animal immune system is devoid of mouse immune cells otherwise represented by human cells.

6. The animal of claim 1, wherein the animal is a NSG mouse.

7. The animal of claim 1, wherein the anti-CCR5 cell receptor binding agent is a monoclonal antibody, protein, or fragment thereof.

8. The animal of claim 7, wherein the monoclonal antibody is PRO 140 or a fragment or conjugate thereof.

9. The animal of claim 6, wherein the mouse has a human CD45+ cell engraftment level after 70 days of greater than about 75% in peripheral blood.

10. The animal of claim 6, wherein the mouse has a human CD45+ cell engraftment level after 70 days of greater than about 65% in bone marrow.

11. The animal of claim 1, wherein the animal substantially maintains or increases in body weight following engraftment.

12. The animal of claim 1, wherein the animal does not exhibit physical symptoms associated with graft-versus-host disease following engraftment.

13. The animal of claim 1, wherein the animal survives at least 70 days following engraftment.

14. The animal of claim 1, wherein the animal survives about two years following engraftment.

15. A method of producing a non-human transgenic animal comprising a humanized immune system and an anti-CCR5 cell receptor binding agent.

16. The method of claim 15, further comprising:
a. selecting an immunocompromised transgenic animal;
b. administering human stem cells to the animal; and
c. administering an anti-CCR5 cell receptor binding agent to the animal.

17. The method of claim 15, further comprising pre-conditioning the transgenic animal.

18. The method of claim 15, wherein the immunocompromised transgenic animal is a mouse.

19. The method of claim 15, wherein the immunocompromised transgenic animal is an NSG mouse.

20. The method of claim 15, wherein the human stem cells are hematopoietic stem cells.

21. The method of claim 15, wherein the anti-CCR5 cell receptor binding agent is a monoclonal antibody, protein, or fragment thereof.

22. The method of claim 21, wherein the anti-CCR5 cell receptor binding agent is PRO 140 or a fragment or conjugate thereof.

23. The method of claim 21, wherein the animal substantially maintains or increases in body weight following engraftment.

24. The method of claim 21, wherein the animal does not exhibit physical symptoms associated with graft-versus-host disease following engraftment.

25. The method of claim 21, wherein the animal survives at least 70 days following engraftment.

26. The method of claim 21, wherein the animal survives about two years following engraftment.

27. A method of producing antibodies for binding to an antigen, comprising immunizing the antigen to a non-human transgenic animal comprising a humanized immune system and an anti-CCR5 cell receptor binding agent.

28. A method of studying a human disease state or condition, comprising using a non-human transgenic animal comprising a humanized immune system and an anti-CCR5 cell receptor binding agent.
